(19) United States
(12) Patent Application Publication
(10) Pub. No.: US 2011/0203960 A1
(43) Pub. Date: Aug. 25, 2011

(54) Cryorganic Massage cosmetic for skin

(75) Inventor: VICTOR GINSZBURG, KFAR SABA (IL)

(73) Assignee: MICRODEL IDEA CENTER, Ramat Gan (IL)

(21) Appl. No.: 12/958,430

(22) Filed: Dec. 2, 2010

Related U.S. Application Data
(60) Provisional application No. 61/307,903, filed on Feb. 25, 2010.

(51) Int. Cl. A61Q 19/00; A61K 8/97; A61K 8/99; A61H 37/00

(52) U.S. Cl. 206/438; 424/635; 424/642; 424/195.16

(57) ABSTRACT

This invention describes the composition of mixtures that are a cryorganic, cryo-massage cream or liquid, for different types of skin, and a method of storing and applying this, non-viscous and thick viscous mixtures, which method can also be used for medications and food products.

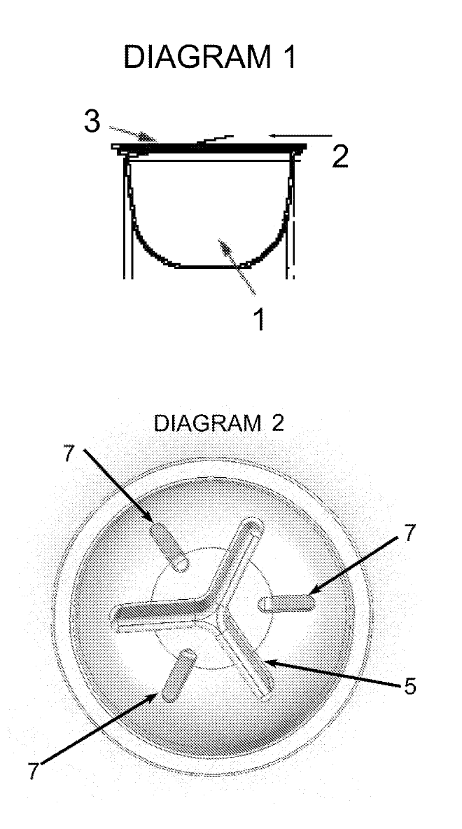

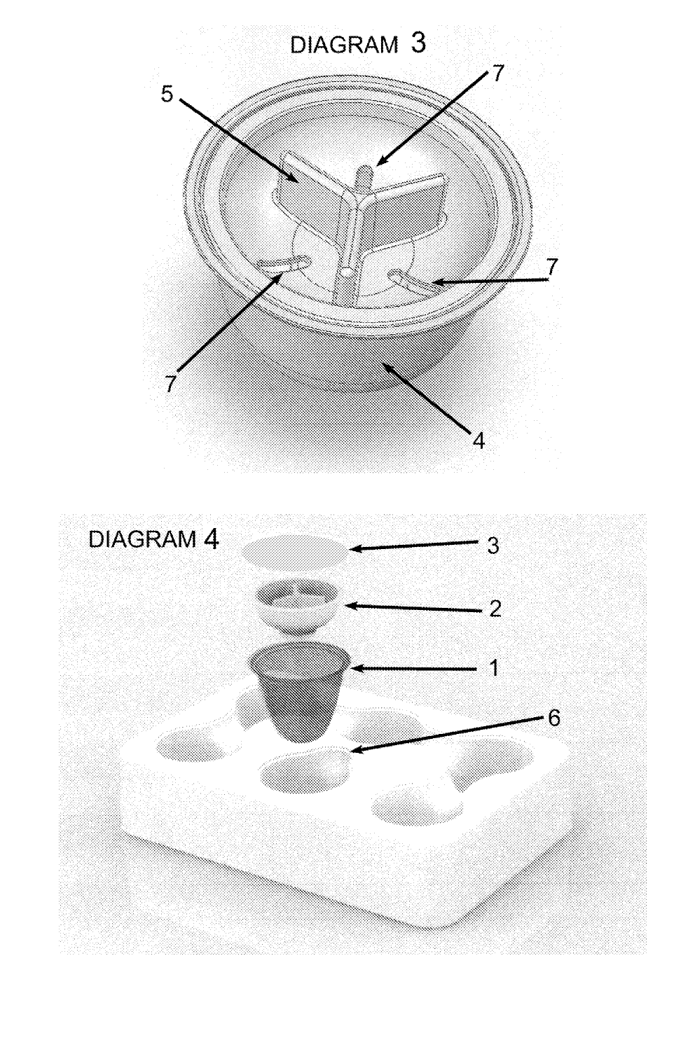

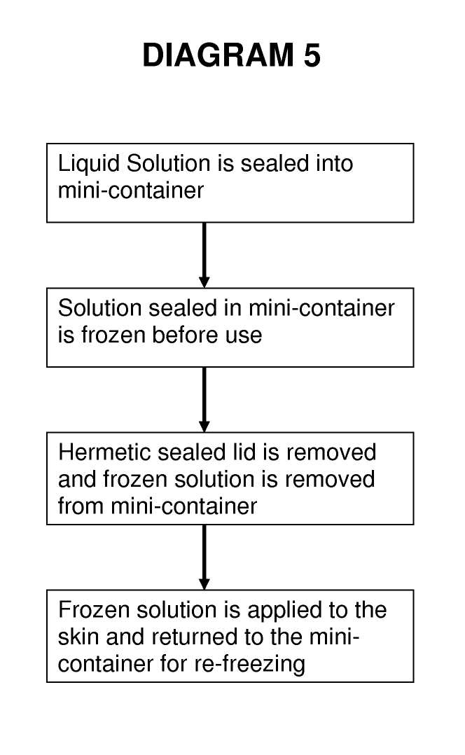

### REFERENCE TO PRIOR APPLICATION

This application claims priority from U.S. Provisional Patent Application No. 61/307,903, filed on Feb. 25, 2010.

### FIELD OF THE INVENTION

This invention has to do with cosmetics, and in particular, with the formulae and method of storing and applying thin, non-viscous and thick, viscous mixtures to the skin. The method can also be used for medications and food products.

### BACKGROUND OF THE INVENTION

Many cosmetic compounds are produced and stored in a frozen state but are thawed to room temperature before use.

Cryo-massage has been known in cosmetics as a rather effective means of applying thin cosmetics in a frozen state (below 0 Celsius) onto the skin with the subsequent thawing on the surface of the skin.

The benefits of such application are further described, which are then followed by the problems, which led to the rather rare use of cryo-cosmetics.

The means of cryo-cosmetic storage are described, including the existing products, along with existing problems and deficiencies of such products and use thereof. The containers described in Israeli patent application No. 109742 of Dr Ginszburg and V Kaganow of 1994 for storing frozen mixtures consist of a body with a cover sealing membrane, where the inner surface of the body is made of a material non-wetted by the mixture and has a smooth surface, and the lid has a rough surface made of material that is adhered to by the mixture. The purpose of the invention described herein is the improvement of the above, which allows for increased efficiency, convenience of use and improved storage of cryo-cosmetics.

### SUMMARY OF THE INVENTION

The stated purpose of the invention is achieved by special composition of the substances, which is optimal for cryo-massage, and which includes zinc [oxide], magnesium [phosphate and aspartate], plant extracts mainly of nettle and jojoba, dissolved in de-ionized (super-soft) water for example in the following % by mass ratios:

0.5-3.0 [zinc]; 0.2-4.0 [nettle]; 0.5-2.8 [jojoba]; with water.

For more effective cryo-massage of oily skin the above basic composition would additionally contain oat kernel extract, cucumber juice and mint.

For more effective cryo-massage of dry or normal skin the basic composition would additionally contain uva ursa, sage and calendula extracts.

For more effective cryo-massage of irritated sensitive skin the basic composition would additionally contain uva ursa, aloe and eucalyptus extracts.

Another class of compositions offered includes, a crème-like base, specifically, as crème hydratant and natural additives of yeast extract, lavender, vitamin B5 (pantothenic acid) and fragrance supplements with the rest being crème hydratant.

Such composition is universal for all types of skin.

For improved cellular permeability of the skin the composition may additionally contain zinc and copper in the ratios of 0.5-7.0 and 0.3-2.5.

This invention also aims to improve mini-containers for the freezing and easy use of the different compositions. A simplified version of containers was outlined in Israeli patent 109742.

### DESCRIPTION OF THE PREFERRED EMBODIMENTS

The following are typical compositions of cryo-cosmetic bases which are proposed for use on different types of skin:

The basic composition for cryo-massage is as follows:

| | |
|---|---|
| Zinc oxide | 0.5-3.0 |
| Nettle extract | 0.2-0.4 |
| Jojoba extract | 0.5-2.8 |

De-ionized water-sufficient to reconstitute to 100%

For oily skin, the following composition is applicable:

Zinc oxide
Magnesium (phosphate and aspartate)
Copper oxide
Nettle extract
Jojoba extract
Oat Kernel extract
Cucumber juice
Mint extract
Uva ursis extract

### Imidazolidinyl Urea

### Glycerin

De-ionized water-sufficient to reconstitute to 100%

For dry/normal skin the following composition is to be used:

Zinc oxide
Magnesium (phosphate and aspartate)
Copper oxide
Nettle extract
Jojoba extract
Orange extract
Sage extract
Calendula extract
Uva ursis extract

### Imidazolidinyl Urea

### Propylene Glycol

### Glycerin

De-ionized water sufficient to reconstitute to 100%

For sensitive/irritated skin the following composition is to be used:

Zinc oxide
Magnesium (phosphate and aspartate)
Copper oxide
Nettle extract
Jojoba extract
Aloe extract
Eucalyptus extract
Orange extract
Uva ursis extract

### Imidazolidinyl Urea

### Propylene Glycol

### Glycerin

De-ionized water-sufficient to reconstitute to 100%

The above examples illustrate the most acceptable combinations of the ingredients. All the ingredients are produced from natural components and are non-allergenic.

Studies conducted on various samples corresponding to the above examples confirmed the importance of the proper balance of ingredients in their application to the particular skin type, such as oily, normal, dry, sensitive, tired skin.

Studies of the samples as per above compositions were conducted according to the methodologies of Dr. R. Voll. The manufacturing methods of the above compositions dissolved in water are obvious and do not require special description.

The composition for cryo-massage on the basis of moisturizing crèmes which is applicable for any skin type, is manufactured according to the following recipe, where ingredients are given in % by mass ratios:

| | |
|---|---|
| Yeast extract | 2-9 |
| Lavender oil | 1-10 |
| Vitamin B5 solution | 0.9-8 |

Crème hydratant base sufficient to reconstitute to 100%

For improved cellular permeability the following ratios apply:

| | |
|---|---|
| Zinc oxide | 0.5-7.0 |
| Copper sulfate | 0.3-2.5 |

Crème hydratant base sufficient to reconstitute to 100%

The studies of the last two compositions have also been conducted according to the methodologies of Dr. Voll. Manufacturing methods of such compositions (on hydratant basis) do not differ from those mentioned above; however, the process of introduction of ingredients has some original technological specifics.

Such specifics lie in the concentrations of the salts (of zinc and copper, 0.5-7.0 and 0.3-2.5%), as well as in the sequence of introduction of these ingredients into the composition (first, the salt of copper [sulfate]; then the salt of zinc [oxide]).

Vital ingredients are introduced into the crème, which regulate skin permeability. According to the infrared spectroscopy data, deep penetration of the dermis takes place within 5-8 minutes on average.

It has to be noted that the compositions offered fully comply with the cosmetic requirements, which are: maintaining the proper aqueous balance, restoration of moisture levels of the dermis of the face and the skin, as well as the prevention of its premature ageing.

The name of “moisturizing crème hydratant” underscores the aforementioned properties of such a crème, which characterizes the more widespread notion of crème base.

### BRIEF DESCRIPTION OF THE DRAWINGS

The following is a legend of the numbering of the parts as illustrated:
1 Container Body
2 Container Lid
3 Hermetic Seal
4 Specially wetted curved surface
5 Holder
6 Hollowed Lip
7 Drainage Hole

Referring now to the drawings Diagram 1 shows the solution offered as far as the mini-container. This is characterized by the fact that it consists of a body (1), which is meant for filling with the liquid mixture (composition) in the form of a solution, emulsion, suspension, or crème-like (paste-like) contents (hereafter called ‘mixture’), the lid (2) for closing the mixture in the body (with partial submersion into the body), and a hermetic seal (3) (plastic film, or aluminum foil, etc.), which is thermally attached to the lid and the body upon it being filled up.

Diagram 2 is a close top view of the lid which has a convex-concave (semi-spherical or conical, etc.) shape, with a triangular shaped holder on the concave side of the lid for easier grasping of the frozen formula. Drainage holes (7) assist in the retention of the frozen mixture when in use.

In Diagram 3 the mushroom like shape of the container is seen. The lid which is immersed into the mixture, has a high wetting property [low surface tension] and adhesion to the frozen mixture due to the properties of the material from which it is made. Wetting is improved due to the special properties of the inner and outer surfaces (4) of the lid—the presence of the holder (5) on the concave side, and draining holes (7).

As seen in Diagram 4, during mass-production the container for the formulae may consist of a block of several mini-containers, separable before the use of each portion of the formula. A hollowed lip (6) would allow for easier removal of each container. Such products, manufactured commercially, may be kept in regular storage for a long time.

Prior to the use of the formula, it would be placed in mini-containers inside the freezer section of a regular refrigerator (at a temperature below 0 C/32 F), where the mixture is frozen and becomes useable. To use the mixture, the hermetic seal (3) needs to be removed, and the lid (2) (with the help of the holder) is then easily removed from the body (1) along with the portion of the mixture frozen to it.

Diagram 5 depicts the process of use and reuse of the mixture. The container with the solution is frozen before use. The frozen contents are extracted from the container for application to the skin, using the handle. The frozen contents are then returned to the container and returned to the freezer for subsequent use.

The container is reusable. Therefore, the combined solution suggested herein, both as mixture composition and container embodiment, offers novelty, usefulness, has inventive steps, and may be manufactured commercially, thus deserving of patent protection.

What is claimed:

1. Composition for cryo-massage including zinc, nettle and jojoba dissolved in distilled water with the following ratio of ingredients given in mass percent:

| | |
|---|---|
| Zinc oxide | 0.5-3.0 |
| Nettle extract | 0.2-0.4 |
| Jojoba extracts | 0.5-2.8 |

De-ionized water: sufficient to reconstitute to 100%

2. Composition, according to claim 1, which for cryo-massage of oily skin additionally contains the following ingredients:
Copper oxide
Magnesium (phosphate and aspartate)
Cucumber juice
Mint extract
Uva-Ursi extract
Oat Kernel extract
Glycerin
Propylene Glycol
Imidazolidinyl Urea

3. Composition, according to claim 1, which for cryo-massage of dry/normal skin additionally contains the following ingredients:
Copper oxide
Magnesium (phosphate and aspartate)
Sage extract
Calendula extract
Uva Ursis extract
Orange Extract
Glycerin
Propylene Glycol
Imidazolidinyl Urea
Propylene Glycol

4. Composition, according to claim 1, which for cryo-massage of sensitive/irritated skin additionally includes the following ingredients:
Aloe extract
Eucalyptus extract
Magnesium (phosphate and aspartate)
Copper oxide
Aloe extract
Orange extract
Uva ursis extract
Imidazolidinyl Urea
Propylene Glycol
Glycerin

5. Composition for cryo-massage including a cream-like basis, in the form of hydratant cream in particular, and natural additives in the form of yeast extract, lavender oil and vitamin B, with the following ratio of ingredients given in mass percent:

| | |
|---|---|
| Yeast extract | 1-9 |
| Lavender oil | 1-10 |
| Vitamin B5 solution | 0.9-8 |

Crème hydratant base sufficient to reconstitute to 100%

6. Composition, according to claim 5, which for improving cells permeability contains fragrance supplements with the following ingredients:
Caprylic Triglyceride
Cetearyl alcohol
Isopropyl mistrate
Panthenol
Propylene Glycol
Ubiquinone
Lavender oil
Cyclomethicone
Imidazolidinyl Urea
Methylparaben
Propylparaben
Tetrasodium Edta
Hexylene Glycol
Urea
Glutamic Acid
Fructose
Alanine
Aspartic Acid
Dextrin
Glucose
Hexyl Nicotinate
Sucrose
Crème hydratant base sufficient to reconstitute to 100%

7. Mini-container packaging for frozen mixture-composition for cryo-massage, including a body, a cover and sealing lid such as a plastic film, whereby the body of the container is made out of the material non-wetted by the frozen mixture and has a smooth inner surface, curved so it will not hinder the extraction of the frozen mixture.

8. Mini-container, as in claim 7 with a cover of a concave-convex shape made of the material wetted and adhering to the frozen mixture, having a holder on the concave surface, and on the convex surface it is fitted with a handle with a surface considerably larger at the end further from the cover.

9. Mini-container, as in claims 7 and 8, with at least one hole in the area next to the center of the cover, and at least one hole on the periphery of the cover for drainage of the air.
